(19) United States
(12) Patent Application Publication
(10) Pub. No.: US 2016/0280638 A1
(43) Pub. Date: Sep. 29, 2016

(54) FILTERING MATERIAL, FILTER, METHOD OF FILTRATION, METHOD OF PRODUCING PHENYLIMINE COMPOUND, METHOD OF PRODUCING ALKOXYPHENYLIMINE COMPOUND, AND COMPOUND

(71) Applicant: TOKYO OHKA KOGYO CO., LTD., Kawasaki-shi (JP)

(72) Inventor: Isao HIRANO, Kawasaki-shi (JP)

(21) Appl. No.: 15/078,887

(22) Filed: Mar. 23, 2016

(30) Foreign Application Priority Data
Mar. 26, 2015 (JP) 2015-064068

(51) Int. Cl. C07C 251/24; C07C 249/02

(52) U.S. Cl. CPC C07C 251/24; C07C 249/02

(57) ABSTRACT

A filtering material including a polymerization product of a compound represented by formula (1-0) or a derivative thereof; and a method of producing a phenylimine compound in which a compound having an aromatic ring and a carbonyl group is reacted with a hydrocarbon compound having at least one amino group, thereby obtaining the phenylimine compound represented by the formula (1-0), the aromatic ring having at least one of a hydroxy group, a thiol group, a carboxy group, a sulfo group, a nitro group and a phosphate group; and a method of producing an alkoxyphenylimine compound in which the phenylimine compound is reacted with an alkylation agent. R¹ represents a polymerizable group-containing hydrocarbon group; R² represents a hydrocarbon group; R³ represents a hydroxy group, a thiol group, a carboxy group, a sulfo group, a nitro group or a phosphate group; and n1 represents 1 to 5.

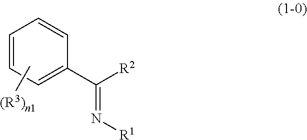

### RELATED APPLICATIONS

This application claims priority to Japanese Patent Application No. 2015-64068, filed Mar. 26, 2015, the content of which is incorporated herein by reference.

### TECHNICAL FIELD

The present invention relates to a filtering material, a filter, a method of filtration, a method of producing phenylimine compound, a method of producing alkoxyphenylimine compound, and a compound.

### BACKGROUND ART

A method of forming a pattern in which a fine pattern is formed on top of a substrate, and a lower layer beneath that pattern is then fabricated by conducting etching with this pattern as a mask are used in the production of semiconductor devices and liquid display device. A lithography method using a resist composition is widely adopted for formation of the fine pattern.

In lithography techniques, a resist film is formed using a resist composition containing a base component such as a resin on a support such as a substrate, and the resist film is subjected to selective exposure of radial rays such as light or electron beam, followed by development, thereby forming a pattern having a predetermined shape on the resist film.

In recent years, further downsize of semiconductor devices have led to rapid progress in the field of pattern miniaturization formed by lithography method. As a pattern is miniaturized, minute defects that were not any problems adversely affect to the lithography properties and becomes a cause of a deteriorated yield in a manufacturing process.

As a cause of the minute defects, metal ions (for example, Fe, Ni, Cr, Na, K and the like) which exist in a resist composition or a solvent used in the pattern formation are known. Also, it was confirmed that even metal ions of less than 100 ppb adversely affect the lithography properties.

In order to solve the above-mentioned problems, attempts have been made to filter and distill the resist composition, thereby removing impurities such as metal ions.

For example, in Patent Document 1 and 2, a method of filtering a resist composition using a filter sheet or the like that uses a functionalized silica gel is disclosed.

In Patent Document 3, a method of removing impurities using impurity filtration apparatus having a polyolefin non-woven having a specific fiber diameter and a specific density as a filtration member is disclosed.

### DOCUMENTS OF RELATED ART

### Patent Literature

[Patent Document 1] Japanese Unexamined Patent Application, First Publication No. 2006-136883

[Patent Document 2] Japanese Unexamined Patent Application, First Publication No. 2003-238958

[Patent Document 3] Japanese Unexamined Patent Application, First Publication No. 2013-61426

### SUMMARY OF THE INVENTION

However, as further miniaturization progresses, a novel filtering material which enables trapping and removing impurities such as metal ions is demanded.

The present invention takes the above circumstances into consideration, with an object of providing a filtering material which enables removing impurities such as metal ions satisfactorily; a filter using the filtering material; a method of filtration using the filter; a method of producing a phenylimine compound which serves as a raw material of the filtering material; a method of producing an alkoxyphenylimine compound using the phenylimine compound; and a compound which serves as a raw material of the filtering material.

For solving the above-mentioned problems, the present invention employs the following aspects.

Specifically, a first aspect of the present invention is a filtering material including a polymerization product of a compound (1-0) represented by general formula (1-0) shown below or a derivative thereof

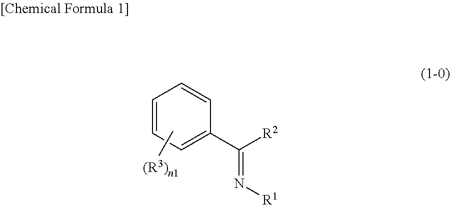

In the formula, R¹ represents a polymerizable group-containing hydrocarbon group which may have a substituent;

R² represents a hydrocarbon group which may have a substituent;

R³ represents a hydroxy group, a thiol group, a carboxy group, a sulfo group, a nitro group or a phosphate group; and

n1 represents an integer of 1 to 5.

A second aspect of the present invention is a filter using the filtering material according to the first aspect.

A third aspect of the present invention is a method of filtration, the method including: passing a resist composition or a solvent through the filter according to the second aspect, and removing impurities contained in the resist composition or the solvent.

The fourth aspect of the present invention is a method of producing a phenylimine compound in which a compound (K) having an aromatic ring and a carbonyl group is reacted with a hydrocarbon compound (A) having at least one amino group, thereby obtaining the phenylimine compound represented by general formula (1-0) shown below, the aromatic ring having at least one group selected from the group consisting of a hydroxy group, a thiol group, a carboxy group, a sulfo group, a nitro group and a phosphate group

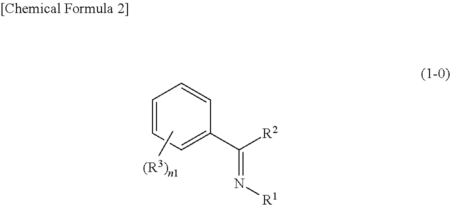

In the formula, R¹ represents a polymerizable group-containing hydrocarbon group which may have a substituent;

R² represents a hydrocarbon group which may have a substituent;

R³ represents a hydroxy group, a thiol group, a carboxy group, a sulfo group, a nitro group or a phosphate group; and

n1 represents an integer of 1 to 5.

A fifth aspect of the present invention is a method of producing an alkoxyphenylimine compound in which the phenylimine compound obtained by the producing method according to the fourth aspect is reacted with an alkylation agent.

A sixth aspect of the present invention is a compound represented by general formula (1-1) shown below.

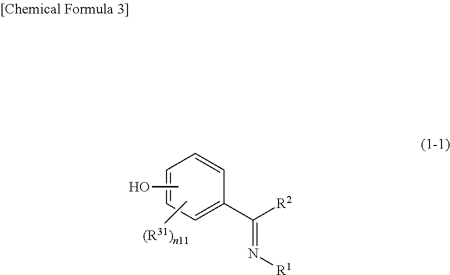

In the formula, R¹ and R² are the same as defined above;

R³¹ represents a hydroxy group, a thiol group, a carboxy group, a sulfo group, a nitro group or a phosphate group; and

n11 represents an integer of 0 to 4.

According to the filtering material, the filter, the method of filtration, the method of producing phenylimine compound, the method of producing alkoxyphenylimine compound and the compound of the present invention, impurities such as metal ions can be satisfactorily removed.

### DETAILED DESCRIPTION OF THE INVENTION

In the present description and claims, the term “aliphatic” is a relative concept used in relation to the term “aromatic”, and defines a group that has no aromaticity or a compound that has no aromaticity.

The term “alkyl group” includes linear, branched or cyclic, monovalent saturated hydrocarbon, unless otherwise specified. The same applies for the alkyl group within an alkoxy group.

The term “alkylene group” includes linear, branched or cyclic, divalent saturated hydrocarbon, unless otherwise specified.

A “halogenated alkyl group” is a group in which part or all of the hydrogen atoms of an alkyl group is substituted with a halogen atom. Examples of the halogen atom include a fluorine atom, a chlorine atom, a bromine atom and an iodine atom.

A “fluorinated alkyl group” or a “fluorinated alkylene group” is a group in which part or all of the hydrogen atoms of an alkyl group or an alkylene group have been substituted with a fluorine atom.

The expression “may have a substituent” means that a case where a hydrogen atom (—H) is substituted with a monovalent group, or a case where a methylene (—CH₂₋) group is substituted with a divalent group.

<Filtering Material>

The filtering material according to the first aspect of the present invention includes a polymerization product of a compound (1-0) represented by general formula (1-0) shown below or a derivative thereof. The polymerization product is a polymeric compound obtained by polymerizing a compound (1-0) represented by general formula (1-0) shown below or a derivative thereof.

The filtering material according to the present invention can be used in filtration of a compound or a composition, and preferably in filtration of a compound or a composition which may contain metal ions as impurities. Particularly, the filtering material is preferably used in filtration of a resist composition which is required to exactly remove impurities and a solvent used in pattern formation or the like.

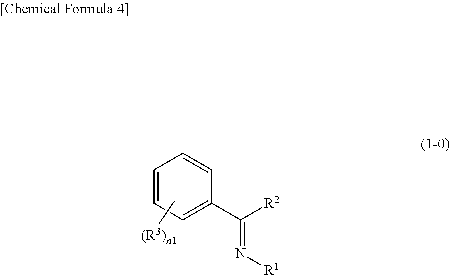

In the formulae, R¹ represents a polymerizable group-containing hydrocarbon group which may have a substituent;

R² represents a hydrocarbon group which may have a substituent;

R³ represents a hydroxy group, a thiol group, a carboxy group, a sulfo group, a nitro group or a phosphate group; and

n1 represents an integer of 1 to 5.

(Compound (1-0))

In formula (1-0), R¹ represents a polymerizable group-containing hydrocarbon group which may have a substituent. A “polymerizable group” refers to a group that renders a compound containing the group polymerizable by a radical polymerization or the like, for example, a group having a carbon-carbon multiple bond such as an ethylenic double bond.

Examples of the polymerizable group include a vinyl group, an allyl group, an acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonylfluorobutylacryloyl group, a vinyl ether group, a fluorine-containing vinyl ether group, an allyl ether group, an fluorine-containing allyl ether group, a styryl group, a vinylnaphthyl group, a fluorine-containing styryl group, a fluorine-containing vinylnaphthyl group, a norbornyl group, a fluorine-containing norbornyl group, and a silyl group.

The polymerizable group-containing hydrocarbon group for R¹ may be a group constituted of only a polymerizable group, or constituted of a polymerizable group and a hydrocarbon group other than a polymerizable group. The hydrocarbon group may have a substituent.

As the polymerizable group-containing hydrocarbon group which may have substituent for R¹, “R¹¹-R¹²” (in the formula, represents a hydrocarbon group containing an ethylenic double bond which may have substituent; and R¹² represents a divalent linking group containing a hetero atom or a single bond, provided that R¹² binds to the nitrogen atom within the formula (1-0)) is preferable.

R¹¹

A hydrocarbon group for R¹¹ is not particularly limited as long as it contains an ethylenic double bond, and may be a chain-like hydrocarbon group, or a hydrocarbon group containing a ring in the structure thereof.

As the chain-like hydrocarbon group for R¹¹, a chain-like alkenyl group is preferable. The chain-like alkenyl group may be linear or branched, and preferably has 2 to 10 carbon atoms, more preferably 2 to 5 carbon atoms, still more preferably 2 to 4 carbon atoms, and particularly preferably 2 or 3 carbon atoms.

Examples of linear alkenyl groups include a vinyl group, a propenyl group (an allyl group) and a butynyl group. Examples of branched alkenyl groups include a 1-methylpropenyl group and a 2-methylpropenyl group. Of these, a vinyl group or a propenyl group is preferable.

Examples of the hydrocarbon group for R¹¹ containing a ring in the structure thereof include an unsaturated aliphatic hydrocarbon cyclic group which contains an ethylenic double bond in the ring structure thereof; a group in which the unsaturated aliphatic hydrocarbon cyclic group is bonded to the terminal of the aforementioned linear or branched aliphatic hydrocarbon group; and a group in which a chain-like alkenyl group is bonded to the terminal of the a cyclic hydrocarbon group.

With respect to “the group in which the unsaturated aliphatic hydrocarbon cyclic group is bonded to the terminal of the aforementioned linear or branched aliphatic hydrocarbon group”, the linear or branched aliphatic hydrocarbon group to which the unsaturated aliphatic hydrocarbon cyclic group is to be bonded may be saturated or unsaturated. In general, the linear or branched aliphatic hydrocarbon group is preferably saturated.

The linear or branched aliphatic hydrocarbon group preferably has 1 to 10 carbon atoms, more preferably 1 to 6, still more preferably 1 to 4, and most preferably 1 to 3.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄-] and a pentamethylene group [—(CH₂)₅—].

As the branched aliphatic hydrocarbon group, branched alkylene groups are preferred, and specific examples include various alkylalkylene groups, including alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. As the alkyl group within the alkylalkylene group, a linear alkyl group of 1 to 5 carbon atoms is preferable.

With respect to the group in which a chain-like alkenyl group is bonded to the terminal of the a cyclic hydrocarbon group, as the chain-like alkenyl group, the same groups as those described above can be mentioned.

The cyclic hydrocarbon group to which the chain-like alkenyl group is to be bonded may be a cyclic aliphatic hydrocarbon group (aliphatic cyclic group) or a cyclic aromatic hydrocarbon group (aromatic cyclic group).

The cyclic aliphatic hydrocarbon group may be either saturated or unsaturated. In general, the cyclic aliphatic hydrocarbon group is preferably saturated.

The aliphatic cyclic group preferably has 3 to 20 carbon atoms, and more preferably 3 to 12 carbon atoms.

The aliphatic cyclic group may be either a monocyclic group or a polycyclic group. As the monocyclic aliphatic hydrocarbon group, a group in which one hydrogen atoms have been removed from a monocycloalkane is preferable. The monocycloalkane preferably has 3 to 6 carbon atoms, and specific examples thereof include cyclopentane and cyclohexane. As the polycyclic aliphatic cyclic group, a group in which 2 hydrogen atoms have been removed from a polycycloalkane is preferable, and the polycyclic group preferably has 7 to 12 carbon atoms. Examples of the polycycloalkane include adamantane, norbornane, isobornane, tricyclodecane and tetracyclododecane.

The aromatic cyclic group is a group in which one hydrogen atom has been removed from an aromatic ring.

The aromatic hydrocarbon ring preferably has 3 to 30 carbon atoms, more preferably 5 to 30, still more preferably 5 to 20, still more preferably 6 to 15, and most preferably 6 to 10. Here, the number of carbon atoms within a substituent(s) is not included in the number of carbon atoms of the aromatic hydrocarbon group.

Examples of the aromatic ring include aromatic hydrocarbon rings, such as benzene, biphenyl, fluorene, naphthalene, anthracene and phenanthrene; and aromatic hetero rings in which part of the carbon atoms constituting the aforementioned aromatic hydrocarbon rings has been substituted with a hetero atom. Examples of the hetero atom within the aromatic hetero rings include an oxygen atom, a sulfur atom and a nitrogen atom.

R¹²

With respect to a divalent linking group containing a hetero atom for R¹², a hetero atom is an atom other than carbon and hydrogen, and examples thereof include an oxygen atom, a nitrogen atom, a sulfur atom and a halogen atom.

In the case where R¹² represents a divalent linking group containing a hetero atom, preferable examples of the linking group include —O—, —C(═O)—O—, —C(═O)—, —O—C(═O)—O—, —C(═O)—NH—, —NH—, —NH—C(═NH)— (wherein H may be substituted with a substituent such as an alkyl group or an acyl group), —S—, —S(═O)₂—, —S(═O)₂—O—, a group represented by general formula —Y²¹—O—Y²²—, —Y²¹—O—, —Y²¹—C(═O)—O—, —C(═O)—O—Y²¹, —C(═O)—NH—Y²¹, —[Y²¹—C(═O)—O]_{m″}—Y²²— or —Y²¹—O—C(═O)—Y²²— [in the formulae, Y²¹ and Y²² each independently represents a divalent hydrocarbon group which may have a substituent; O represents an oxygen atom; and m″ represents an integer of 0 to 3].

The divalent linking group containing a hetero atom represents —C(═O)—NH—, —NH—, or —NH—C(═NH)—, —C(═O)—NH—Y²¹, H may be substituted with a substituent such as an alkyl group, an acyl group or the like. The substituent (an alkyl group, an acyl group or the like) preferably has 1 to 10 carbon atoms, more preferably 1 to 8, and most preferably 1 to 5.

In formulae —Y²¹—O—Y²²—, —Y²¹—O—, —Y²¹—C(═O)—O—, —C(═O)—O—Y²¹—, —C(═O)—NH—Y²¹, —[Y²¹—C(O)—O]_{m″}—Y²²— and —Y²¹—O—C(═O)—Y²²—, Y²¹ and Y²² each independently represents a divalent hydrocarbon group which may have a substituent.

As Y²¹, a linear aliphatic hydrocarbon group is preferable, more preferably a linear alkylene group, still more preferably a linear alkylene group of 1 to 5 carbon atoms, and a linear alkylene group of 1 to 3 carbon atoms is particularly desirable.

As Y²², a linear or branched aliphatic hydrocarbon group is preferable, and a methylene group, an ethylene group or an alkylmethylene group is more preferable. The alkyl group within the alkylmethylene group is preferably a linear alkyl group of 1 to 5 carbon atoms, more preferably a linear alkyl group of 1 to 3 carbon atoms, and most preferably a methyl group.

In the group represented by the formula —[Y²¹—C(═O)—O]_{m″}Y²²—, m″ represents an integer of 0 to 3, preferably an integer of 0 to 2, more preferably 0 or 1, and most preferably 1. Namely, it is particularly desirable that the group represented by the formula —[Y²¹—C(═O)—O]_{m″}—Y²²— is a group represented by the formula —Y²¹—C(═O)—O—Y²²—. Among these, a group represented by the formula —(CH₂)ₐ—C(═O)—O—(CH₂)_{b′}— is preferable. In the formula, a′ is an integer of 1 to 10, preferably an integer of 1 to 8, more preferably an integer of 1 to 5, still more preferably 1 or 2, and most preferably 1. b′ is an integer of 1 to 10, preferably an integer of 1 to 8, more preferably an integer of 1 to 5, still more preferably 1 or 2, and most preferably 1.

Among them, as R¹, a group represented by “R¹¹-R¹²” (R¹¹ and R¹² are the same as defined above) is preferable; a group consisting of a chain-like alkenyl group, a group in which a chain-like alkenyl group is bonded to the terminal of the a cyclic hydrocarbon group, or a combination of a chain-like alkenyl group and a divalent linking group containing a hetero atom (more preferably, a group containing —C(═O)—NH—) is more preferable.

In formula (1-0), R² represents a hydrocarbon group which may have a substituent.

Examples of the hydrocarbon group for R² include a linear or branched alkyl group and a cyclic hydrocarbon group.

The linear alkyl group preferably has 1 to 5 carbon atoms, more preferably 1 to 4, and still more preferably 1 or 2. Specific examples include a methyl group, an ethyl group, an n-propyl group, an n-butyl group and an n-pentyl group. Among these, a methyl group, an ethyl group or an n-butyl group is preferable, and a methyl group or an ethyl group is more preferable.

The branched alkyl group preferably has 3 to 10 carbon atoms, and more preferably 3 to 5. Specific examples include an isopropyl group, an isobutyl group, a tert-butyl group, an isopentyl group, a neopentyl group a 1,1-diethylpropyl group and a 2,2-dimethylbutyl group. Among these, an isopropyl group is preferable.

The linear or branched alkyl group may or may not have a substituent. Examples of the substituent include a fluorine atom, a fluorinated alkyl group of 1 to 5 carbon atoms, a hydroxy group, a thiol group, a carboxy group, a sulfo group, a nitro group and a phosphate group.

In the case where R² represents a cyclic hydrocarbon group, the cyclic hydrocarbon group may be an aliphatic hydrocarbon group or an aromatic hydrocarbon group, and may be polycyclic or monocyclic.

As the monocyclic aliphatic hydrocarbon group, a group in which 1 hydrogen atoms have been removed from a monocycloalkane is preferable. The monocycloalkane preferably has 3 to 6 carbon atoms, and specific examples thereof include cyclopentane and cyclohexane.

As the polycyclic aliphatic hydrocarbon group, a group in which 1 hydrogen atoms have been removed from a polycycloalkane is preferable, and the polycyclic group preferably has 7 to 12 carbon atoms. Examples of the polycycloalkane include adamantane, norbornane, isobornane, tricyclodecane and tetracyclododecane.

When the cyclic hydrocarbon group for R² is an aromatic hydrocarbon group, the aromatic hydrocarbon group is a hydrocarbon group having at least one aromatic ring.

The aromatic ring is not particularly limited, as long as it is a cyclic conjugated compound having (4n+2) π electrons, and may be either monocyclic or polycyclic. The aromatic ring preferably has 5 to 30 carbon atoms, more preferably 5 to 20, still more preferably 6 to 15, and most preferably 6 to 12. Examples of the aromatic ring include aromatic hydrocarbon rings, such as benzene, naphthalene, anthracene and phenanthrene; and aromatic hetero rings in which part of the carbon atoms constituting the aforementioned aromatic hydrocarbon rings has been substituted with a hetero atom. Examples of the hetero atom within the aromatic hetero rings include an oxygen atom, a sulfur atom and a nitrogen atom. Specific examples of the aromatic hetero ring include pyridine ring, thiophene ring, and the like.

Specific examples of the aromatic hydrocarbon group for R² include a group in which one hydrogen atom has been removed from the aforementioned aromatic hydrocarbon ring or aromatic hetero ring (aryl group or heteroaryl group); a group in which one hydrogen atom has been removed from an aromatic compound having two or more aromatic rings (biphenyl, fluorene or the like); and a group in which one hydrogen atom of the aforementioned aromatic hydrocarbon ring or aromatic hetero ring has been substituted with an alkylene group (an arylalkyl group such as a benzyl group or a phenethyl group). The alkylene group bonded to the aforementioned aromatic hydrocarbon ring or the aromatic hetero ring preferably has 1 to 4 carbon atoms, more preferably 1 or 2 carbon atoms, and most preferably 1 carbon atom.

The cyclic hydrocarbon group for R² may or may not have a substituent. Examples of substituents include an alkyl group, an alkoxy group, a halogen atom, a halogenated alkyl group, a hydroxy group, a thiol group, a carboxy group, a sulfo group, a nitro group and a phosphate group.

The alkyl group as the substituent is preferably an alkyl group of 1 to 5 carbon atoms, and a methyl group, an ethyl group, a propyl group, an n-butyl group or a tert-butyl group is particularly desirable.

The alkoxy group as the substituent is preferably an alkoxy group having 1 to 5 carbon atoms, more preferably a methoxy group, ethoxy group, n-propoxy group, iso-propoxy group, n-butoxy group or tert-butoxy group, and most preferably a methoxy group or an ethoxy group.

Examples of the halogen atom for the substituent include a fluorine atom, a chlorine atom, a bromine atom and an iodine atom, and a fluorine atom is preferable.

Examples of the halogenated alkyl group for the substituent include groups in which part or all of the hydrogen atoms within the aforementioned alkyl groups has been substituted with the aforementioned halogen atoms.

In the case where R² is an aromatic hydrocarbon group (a group in which 1 hydrogen atom has been removed from an aromatic hydrocarbon ring) having a substituent, examples include a 1-naphthylmethyl group, a 2-naphthylmethyl group, a 1-naphthylethyl group, or a 2-naphthylethyl group.

Among them, as R², a linear or branched alkyl group of 1 to 5 carbon atoms which may have a substituent, or an aromatic hydrocarbon group which may have a substituent is preferable; a linear alkyl group of 1 to 3 carbon atoms which may have a substituent, or an aromatic hydrocarbon group which may have a substituent is more preferable; a methyl group, an ethyl group or a phenyl group which may have a substituent is still more preferable; and a phenyl group which may have a substituent is desirable.

As a substituent of the phenyl group, a hydroxy group, a thiol group, a carboxy group, a sulfo group, a nitro group and a phosphate group are preferable, and a hydroxy group is desirable. The phenyl group may have a plurality of substituents, and the plurality of substituents may be the same or different from each other.

That is, as the compound (1-0) represented by formula (1-0), a compound represented by formula (1-2) shown below is preferable.

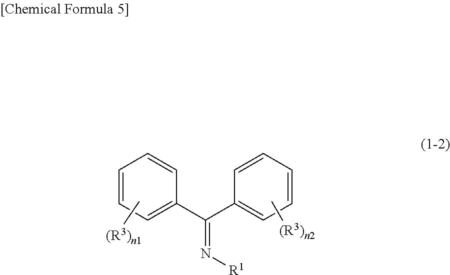

In the formula, R¹, R³ and n1 are the same as defined above, provided that the plurality of R³ may be the same or different from each other; and

n2 represents an integer of 0 to 5.

In formula (1-2), R³ and n1 are the same as R³ and n1 described below. n2 represents an integer of 0 to 5, preferably 0 to 2 and more preferably 0 or 1.

In the aforementioned formula (1-0), R³ represents a hydroxy group, a thiol group, a carboxy group, a sulfo group, a nitro group or a phosphate group. One or more of R³ are preferably a hydroxy group.

At least one R³ is preferably bonded to the para position of the imino group.

n1 represents an integer of 1 to 5, preferably 1 to 3, and most preferably 1 or 2.

That is, as the compound (1-0) represented by formula (1-0), a compound represented by formula (1-1) shown below and a compound represented by formula (1-3) shown below are also preferable.

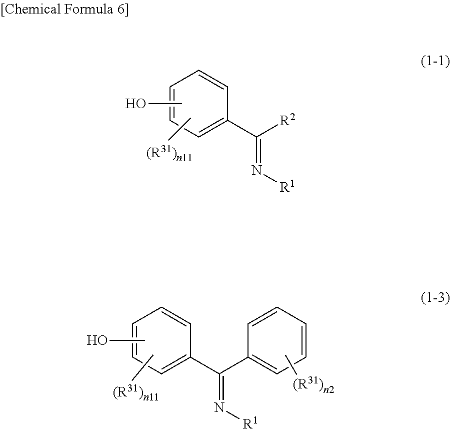

In formulae (1-1) and (1-3), R¹ is the same as defined above.

In formula (1-1), R² is the same as defined above.

In formulae (1-1) and (1-3), R³¹ represents a hydroxy group, a thiol group, a carboxy group, a sulfo group, a nitro group or a phosphate group, and preferably a hydroxy group. A plurality of R³¹ may be the same or different from each other.

In formulae (1-1) and (1-3), n11 represents an integer of 0 to 4, preferably an integer of 0 to 2, and still more preferably 0 or 1.

In formula (1-3), n2 represents an integer of 0 to 5, and preferably 0 or 1.

Specific examples of the compound (1-0) are shown below.

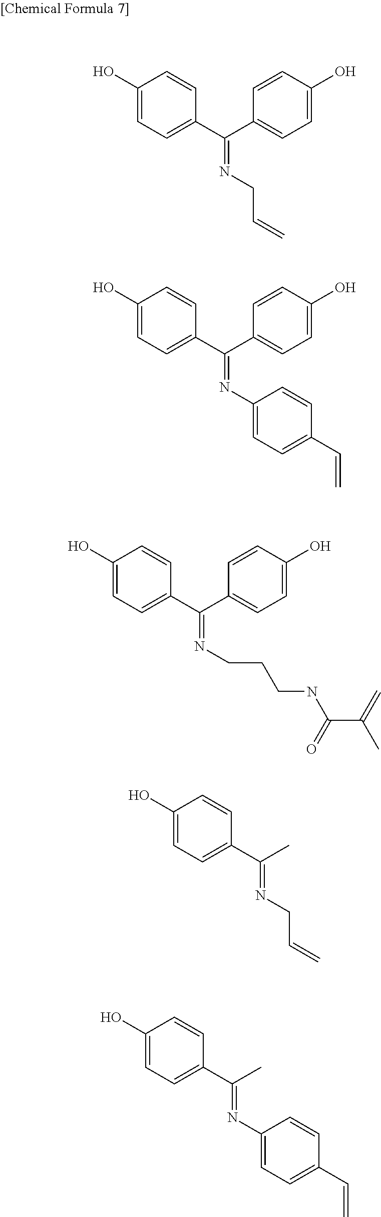 [SMILES: C=CCN=C(c1ccc(O)cc1)c1ccc(O)cc1 ; C=Cc1ccc(N=C(c2ccc(O)cc2)c2ccc(O)cc2)cc1 ; C=C(C)C(=O)NCCCN=C(c1ccc(O)cc1)c1ccc(O)cc1 ; C=CC/N=C(\C)c1ccc(O)cc1 ; C=Cc1ccc(/N=C(\C)c2ccc(O)cc2)cc1]

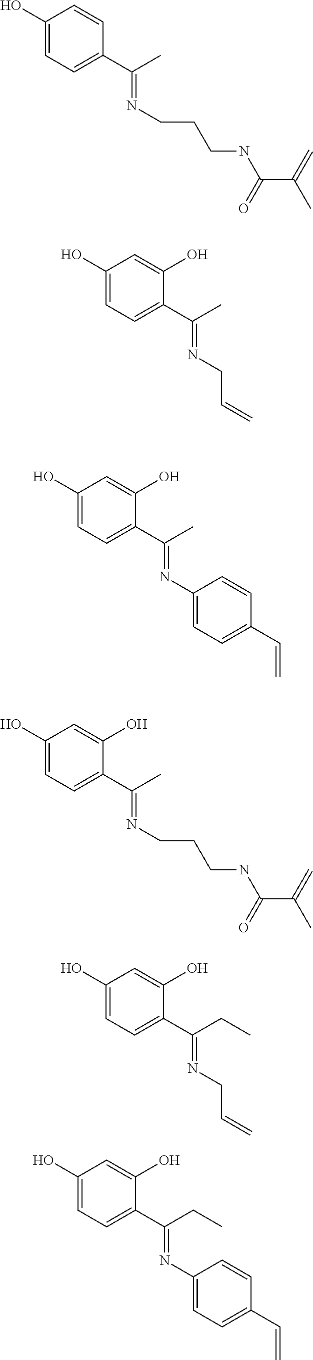 [SMILES: C=C(C)C(=O)NCCC/N=C(\C)c1ccc(O)cc1 ; C=CC/N=C(\C)c1ccc(O)cc1O ; C=Cc1ccc(/N=C(\C)c2ccc(O)cc2O)cc1 ; C=C(C)C(=O)NCCC/N=C(\C)c1ccc(O)cc1O ; C=CC/N=C(\CC)c1ccc(O)cc1O ; C=Cc1ccc(/N=C(\CC)c2ccc(O)cc2O)cc1]

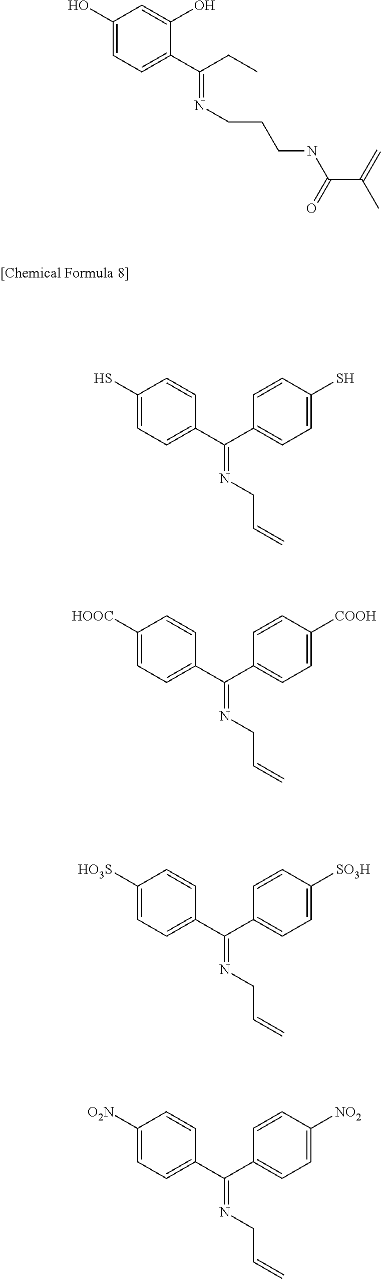 [SMILES: C=C(C)C(=O)NCCC/N=C(\CC)c1ccc(O)cc1O ; C=CCN=C(c1ccc(S)cc1)c1ccc(S)cc1 ; C=CCN=C(c1ccc(C(=O)O)cc1)c1ccc(C(=O)O)cc1 ; C=CCN=C(c1ccc(S(=O)(=O)O)cc1)c1ccc(S(=O)(=O)O)cc1 ; C=CCN=C(c1ccc([N+](=O)[O-])cc1)c1ccc([N+](=O)[O-])cc1]

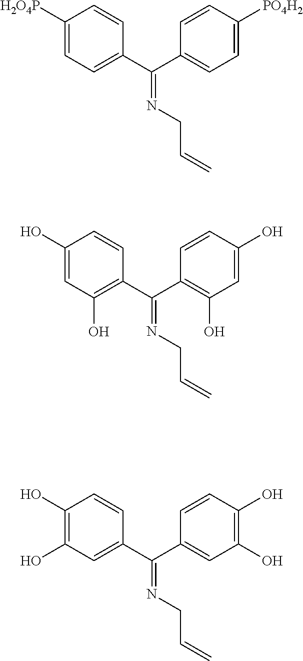

In the present aspect, a “derivative of compound (1-0)” means a compound in which part or all of the hydrogen atoms within the compound (1-0) have been substituted with a substituent other than a hydrogen atom.

Examples of the substituent other than a hydrogen atom include an alkyl group, a halogen atom and a halogenated alkyl group, and they are the same as defined for the alkyl group, the halogen atom and the halogenated alkyl group as the substituent for the cyclic hydrocarbon group of R². Among these, a derivative in which a hydrogen atom within the compound (1-0) is substituted with an alkyl group is preferably used; and a derivative in which a hydrogen atom of R³ within the compound (1-0) is substituted with an alkyl group is still more preferably used.

(Polymerization of Compound (1-0) or Derivative Thereof)

The filtering material according to the first aspect of the present invention uses a polymeric compound (polymerization product) obtained by polymerizing the compound (1-0) or a derivative thereof. The compound (1-0) or the derivative thereof has a polymerizable group in R¹ within the structure thereof, and thus the compound (1-0) can be mutually polymerized by a conventional method.

The compound (1-0) or the derivative thereof can be polymerized, for example, by a conventional radical polymerization or the like, using a radical polymerization initiator such as azobisisobutyronitrile (AIBN) or dimethyl 2,2′-azobis(isobutyrate).

The filtering material of the present invention has an imino group which enables trapping metals by forming a metal complex. By virtue of using the filtering material, impurities, especially metal components such as metal ions, can be efficiently removed. When the object to be filtered is a resist composition, impurities (trace metal tiny particles, trace metals, trace metal ions or the like) within an organic solvent used in the resist composition can be removed. These metal components may be originally contained in the organic solvent, or may be inmixed by contamination from a transport pathway of the organic solvent such as pipework or joints, and thus it has been difficult to remove these metal components.

According to the filtering material of the present invention, metals such as lithium, sodium, magnesium, potassium, chromium, manganese, iron, cobalt, nickel, copper, zinc, strontium, molybdenum, silver, cadmium, tin, antimony, barium, lead, and the like and the ions thereof can be removed. By the filtering material of the present invention, even in the case where two or more kinds of these metal components are present in combination, they can be removed.

Particularly, a metal component having high ionization tendency, or iron, chromium and nickel which are likely to be inmixed by contamination during the manufacturing process can be effectively removed.

<Filter>

The filter according to the second aspect of the present invention uses the filtering material according to the first aspect.

The shape of the filter is not particularly limited. A flat shape, a roll shape, a cone shape, a pleated shape, a spiral shape, a layered type, or a combination of these may be used, and a flat shape or a roll shape is preferable. However, as described in the third aspect, the filter according to the present aspect is aimed to be used by passing an object to be filtered through the filter. Thus, in terms of convenience and filtering property during solution passage, the filter is preferably a shape having high-specific surface area, and desirably a flat shape.

Furthermore, the filter having a flat shape may be used, for example, as cut disk having a diameter of 20 mm to 300 mm.

Moreover, the filter of the present invention may be cartridge type. As the cartridge type filter, for example, a cartridge device which is formed as one or more layers, and has a pleats or is wound up spirally is preferable. Further, a cartridge device having a flat shape and sheet shape is more preferable.

<Method of Filtration>

The method of filtration according to the third aspect of the present invention includes: passing a resist composition or a solvent through the filter according to the second aspect, and removing impurities contained in the resist composition or the solvent.

The filtration can be performed, for example, by setting up the filter according to the second aspect on a filter cartridge or a column, and by sending solution, if desired, using a conventional filtration apparatus.

In the method of filtration according to the present invention, a resist composition and a solvent as the object to be filtered is not particularly limited, and the method of filtration according to the present invention can be applied to a conventional resist composition and solvent.

For example, the resist composition may be either a non-chemically amplified resist composition or a chemically amplified resist composition. Examples of non-chemically amplified resist composition include a composition containing a novolac resin, a photosensitizer, and an organic solvent. Examples of chemically amplified resist composition include a composition containing a resin which exhibits changed solubility in a developing solution under action of acid, an acid generator and an organic solvent.

Examples of the solvent includes solutions such as a thinner, a resist developing solution, a resist stripping solution, an insulation material, ARC (Anti Reflective Coating); ultra-pure water as a cleaning solution, an organic solvent, a mixed aqueous solution of ammonia and hydrogen peroxide, a dilute hydrofluoric acid (DHF) solution, a buffered hydrofluoric acid (BHF) solution, and the like.

Examples of the organic solvent includes lactones, ketones, polyhydric alcohols, derivatives of polyhydric alcohols, cyclic ethers, esters, aromatic organic solvents, nitrile-based organic solvents, and the like.

The flow rate of the resist composition or the solvent during passing through the filter hardly affect the efficiency of metal separation, and is generally set within a range of 0.0001 to 1000 kg/(m²·min). When the temperature of the solution passing through the filter is too high, elution or deterioration of filtering medium or degradation of the resist composition or the solution is likely to occur. Further, when the temperature is too low, in the case of the resist composition, viscosity of the resin within the solution increases, and thus it becomes difficult to allow the solution to pass through the filter. Therefore, the range of the temperature is preferably set within a range of 0 to 50° C.

In the method of filtration according to the present invention, a metal component as an impurity can be preferably removed.

By the method of filtration according to the present invention, metal components such as lithium, sodium, magnesium, potassium, chromium, manganese, iron, cobalt, nickel, copper, zinc, strontium, molybdenum, silver, cadmium, tin, antimony, barium, lead, and the like can be removed.

By the method of filtration according to the present invention, even in the case where two or more kinds of these metal components are present in combination, they can be removed.

Among them, a metal component having high ionization tendency, or iron, chromium and nickel which are likely to be inmixed by contamination during the manufacturing process can be effectively removed.

By the method of filtration according to the present invention, aforementioned trace metals can be removed regardless of the form in which they are present, such as metal ions, metal tiny particles, or the like.

The method of filtration according to the present invention uses the filter according to the present invention with replacing or adding to a conventional filter cartridge provided so as to remove tiny-particle impurities in supply line of various chemicals or POU (Point of Use) of the semiconductor manufacturing process. Therefore, the method of filtration enables efficient removing of tiny particle impurities and trace metal impurities at the same time using a conventional apparatus and conventional operation.

That is, by achieving removal of trace metal impurities in single filtration process, the present invention can be easily applied to real devices currently used in the semiconductor device manufacturing, and thus the benefits made by the present invention to the semiconductor industry is numerous.

Further, when the method of filtration according to the present invention is performed during the circulation pathway of chemical tanks in the supply line of various chemicals (solutions) provided at semiconductor device manufacturing process, metal impurities and tiny particle impurities contained in various chemicals can be effectively removed. Further, in addition to removal of metal impurities which was originally contained in chemicals, pollution from chemical transporting pathway such as pipework or joints can be handled.

<Method of Producing Phenylimine Compound>

In the fourth aspect of the present invention, a phenylimine compound represented by formula (1-0) is obtained by reacting a compound (K) having an aromatic ring and a carbonyl group with a hydrocarbon compound (A) having at least one amino group, the aromatic ring having at least one group selected from the group consisting of a hydroxy group, a thiol group, a carboxy group, a sulfo group, a nitro group and a phosphate group.

By virtue of adopting the method and producing a phenylimine compound, the objective compound can be produced with an extremely high yield, without using an acid catalyst, just by stirring.

The phenylimine compound obtained by the fourth aspect of the present invention can be used in the filtering material according to the first aspect of the present invention. The phenylimine compound represented by formula (1-0), and R¹ to R³ and n1 in the formula (1-0) are the same as defined above.

That is, in the method of producing a phenylimine compound according to the present aspect, a ketone compound (K) which has an aromatic ring having a substituent and a carbonyl group and an amine compound (A) which has at least one amino group are dissolved in an appropriate organic solvent, and the reaction is performed by stirring while cooling, thereby obtaining the phenylimine compound.

Particularly, as shown in the following formula, a carbonyl group within the ketone compound (K0) is subjected to a dehydration/condensation reaction with an amino group within the amine compound (A1) in an appropriate organic solvent, thereby obtaining the phenylimine compound (1-0). More preferably, a carbonyl group within the ketone compound (K1) having a hydroxy group is subjected to a dehydration/condensation reaction with the amino group within the amine compound (A1) in an appropriate organic solvent, thereby obtaining the hydrogen group-containing phenylimine compound (1-1).

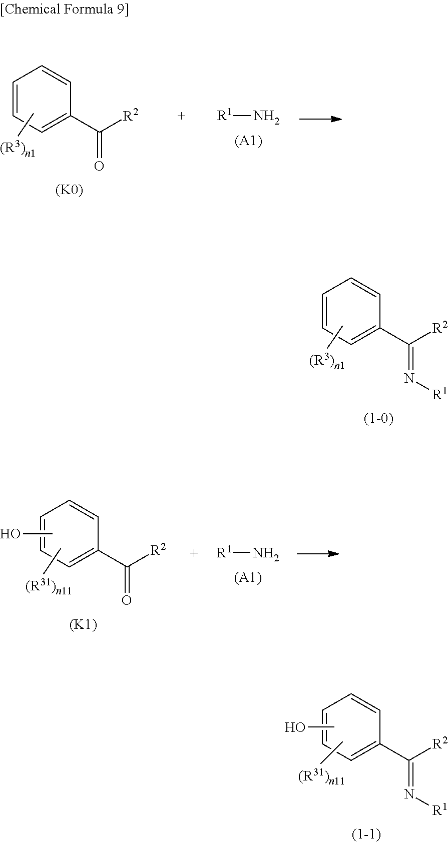

In the formulae, R¹ to R³, R³¹, n1, n11, and the compound (1-0) and (1-1) are the same as defined in the first aspect.

Examples of the ketone compound (K), as represented by formula (K0), include 2,4-dihydroxy acetophenone, 4-hydroxyacetophenone, 4-hydroxybenzophenone, 2,4-dihydroxypropiophenone, 2,4-dihydroxybenzophenone, 2,3,4-trihydroxyacetophenone, 2,3′,4,4′-tetrahydroxybenzophenone and the like.

Further, examples of the amine compound (A), as represented by formula (A1), include p-amino styrene and N-aminoisopropyl methacrylamide.

The reaction temperature is preferably 10° C. or lower, and more preferably 0° C. or lower. The reaction time is preferably 0.5 to 48 hours, and more preferably 1 to 36 hours.

The organic solvent is not particularly limited as long as the dehydration/condensation reaction proceeds and dehydrated ethanol is preferable.

The amount of the raw material compound is preferably the ketone compound (K): the amine compound (A)=1:2 to 2:1 (molar ratio), and more preferably 1:1.5 to 1.5:1 (molar ratio), and desirably 1:1 (molar ratio).

<Method of Producing Alkoxyphenylimine Compound>

The fifth aspect of the present invention is a method of producing an alkoxyphenylimine compound in which the phenylimine compound obtained by the producing method according to the fourth aspect is reacted with an alkylation agent.

That is, in the fifth aspect, the hydroxy group, the thiol group, the carboxy group, the sulfo group, the nitro group or the phosphate group for the substituent R³ within the phenylimine compound represented by aforementioned formula (1-0) is alkylated, thereby obtaining an alkoxyphenylimine compound.

The alkoxyphenylimine compound obtained in the fifth aspect is a compound which corresponds to a derivative of the phenylimine compound represented by formula (1-0) and can be used for the filtering material according to the first aspect of the present invention.

For example, as shown in the following formula, a phenylimine compound is reacted with an alkylation agent in an appropriate solvent and a hydrogen atom of a hydroxy group, a thiol group, a carboxy group, a sulfo group, a nitro group or a phosphate group within the phenylimine compound is substituted with an alkyl group, thereby obtaining an alkoxyphenylimine compound.

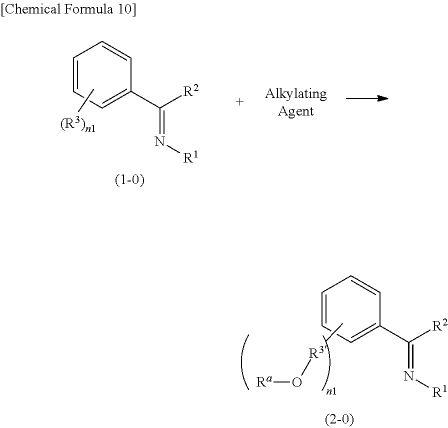

In the formulae, R¹ to R³, n1 and the compound (1-0) are the same as defined for R¹ to R³ and the compound (1-0) of the first aspect. R^{3′} is a group in which a hydrogen atom has been removed from a hydroxy group, a thiol group, a carboxy group, a sulfo group, a nitro group or a phosphate group, and IV is an alkyl group of 1 to 5 carbon atoms derived from an alkylation agent.

More specifically, an aqueous solution of sodium hydroxide or the like is added to the phenylimine compound in order to form a sodium salt and be soluble in water, and reaction is performed by adding the alkylation agent while stirring, thereby forming an objective alkoxyphenylimine compound.

The reaction temperature is preferably 10° C. or lower, and more preferably 0° C. or lower. The reaction time is preferably 0.5 to 48 hours, and more preferably 1 to 36 hours.

As the alkylation agent, one or more selected from the group consisting of dimethylsulfate, iodomethane, dimethylcarbonate and diethylsulfate is preferable.

The amount of the alkylation agent is preferably 1 to 10 moles, and more preferably 1 to 5 moles, per 1 mole of the phenylimine compound.

The structure of the compound obtained after each of the steps described above can be confirmed by a general organic analysis method such as ¹H-nuclear magnetic resonance (NMR) spectrometry, ¹³C-NMR spectrometry, ¹⁹F-NMR spectrometry, infrared absorption (IR) spectrometry, mass spectrometry (MS), elementary analysis and X-ray diffraction analysis.

<Novel Compound>

The sixth aspect of the present invention is a compound represented by general formula (1-1).

In the formula, R¹, R², R³¹ and n11 are the same as defined above for R¹, R², R³¹ and n11 of the first aspect.

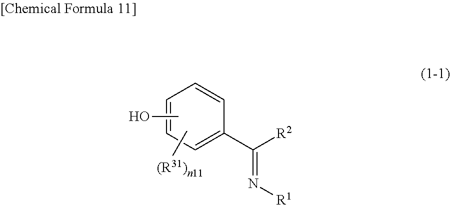

In the formula, R¹ and R² are the same as defined above;

R³¹ represents a hydroxy group, a thiol group, a carboxy group, a sulfo group, a nitro group or a phosphate group; and

n11 represents an integer of 0 to 4.

### Examples

As follows is a description of examples of the present invention, although the scope of the present invention is by no way limited by these examples.

### Example 1

### Production of 2-propen-1-amine, N-[bis(4-dihydroxyphenyl) methylene]

119.52 g, (0.697 mol) of 4,4′-dihydroxybenzophenone was added to a recovery flask, and 400 ml of dehydrated ethanol was further added thereto and dissolved. Then, 40 g (0.697 mol) of allylamine was added thereto while cooling in an ice bath, followed by stirring at 0° C. for 2 hours. The dehydration/condensation reaction with allylamine caused imination, thereby immediately changing colorless solution to yellow solution.

Thereafter, ethanol and water generated by the reaction was distilled off from the resulting solution using an evaporator, and then the resultant was dried in vacuum all night and all day, thereby obtaining 2-propen-1-amine, N-[bis(4-dihydroxyphenyl) methylene].

The obtained compound was a yellow solid, and the yield was 91%. The compound was soluble in ethanol, acetone and DMF, and insoluble in water, hexane and dichloromethane.

The obtained compound was analyzed by NMR.

¹H-NMR (Acetone): 9.95 (s, OH), 7.41-6.77 (m, 8H), 6.02-5.98 (m, 2H), 5.27-5.15 (m, 1H), 4.20 (d, 2H).

From the results shown above, it was confirmed that the compound had a structure as shown below.

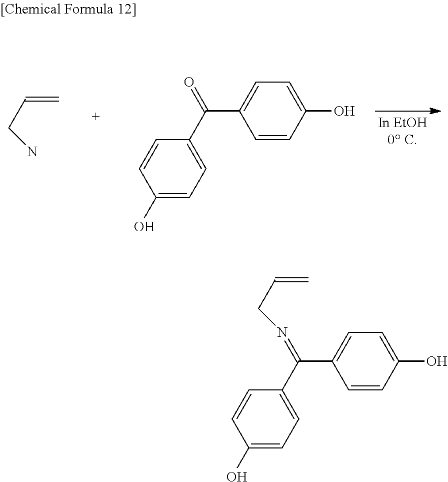

### Example 2

### Synthesis of 2-propen-1-amine, N-[bis(4-methoxy-phenyl)methylene]

36.29 g (0.1289 mol) of 2-propen-1-amine, N-[bis(4-dihydroxyphenyl) methylene] obtained in above Example 1 was added to a conical flask, and 600 mL of 1 mol/L sodium hydroxide was further added thereto while stirring and cooling in an ice bath at 0° C. While stirring, a hydroxy group within 2-propen-1-amine, N-[bis (4-dihydroxyphenyl) methylene] reacted with sodium ion, thereby forming a sodium alkoxide salt of 2-propen-1-amine, N-[bis(4-dihydroxyphenyl) methylene]. The salt was dissolved in water and the solution changed to bright yellow.

The solution was further added 61.04 g (0.556 mol) of dimethyl sulfate and stirred for 2 hours while continuously cooling in an ice bath at 0° C. The sodium alkoxide salt of 2-propen-1-amine, N-[bis(4-dihydroxyphenyl)methylene] reacted with dimethyl sulfate, thereby forming 2-propen-1-amine, N-[bis(4-methoxy-phenyl)methylene]. The formed compound was water-insoluble, and thus a solid was precipitated in the solution.

After completion of the reaction, the precipitate was collected by filtration under reduced pressure and washed with saturated sodium hydrogen carbonate aqueous solution to remove by-produced N-allylmethylamine or the like, followed by washing with hexane.

Thereafter, the objective product of 2-propen-1-amine, N-[bis (4-methoxy-phenyl) methylene] was obtained by drying all day and all night.

The obtained compound was a pale yellow solid, and the yield was 75%. The compound was soluble in methanol, ethanol, acetone and dichloromethane, and insoluble in water.

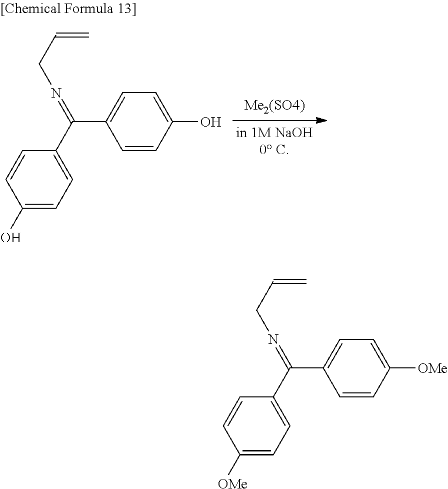 [SMILES: C=CCN=C(c1ccc(O)cc1)c1ccc(O)cc1 ; C=CCN=C(c1ccc(OC)cc1)c1ccc(OC)cc1]

### Example 3

### Metal Trapping Test

A metal trapping test was conducted using 2-propen-1-amine, N-[bis (4-methoxy-phenyl) methylene] (hereafter, referred to as “filtering material”) obtained in above Example 2.

Particularly, 4 ml of FeCl₃ solution in ethanol was dropwise added to 1 g of the filtering material according to Example 2. The FeCl₃ solution in ethanol before filtration was yellow. After stirring for 5 minutes, the filtering material was removed by filtration. The removed filtering material was reddish, and the filtrate was colorless. As the filtrate was colorless, it was confirmed that metal components were completely absorbed.

Therefore, it was confirmed that metal components such as irons or the like which exist in solution was trapped and removed with high efficiency by using the filtering material according to the present invention.

An organic solution constitutes 90% by weight or more of a resist composition. Therefore, from the above result that metal components can be removed from an organic solvent with high efficiency, the filtering material according to the present invention can remove metal components in a resist composition with high efficiency.

What is claimed is:

1. A filtering material comprising a polymerization product of a compound (1-0) represented by general formula (1-0) shown below or a derivative thereof: 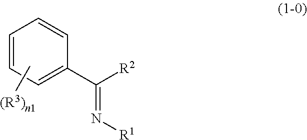
wherein R¹ represents a polymerizable group-containing hydrocarbon group optionally having a substituent; R² represents a hydrocarbon group optionally having a substituent; R³ represents a hydroxy group, a thiol group, a carboxy group, a sulfo group, a nitro group or a phosphate group; and n1 represents an integer of 1 to 5.

2. The filtering material according to claim 1, wherein the compound (1-0) is represented by general formula (1-1) shown below: 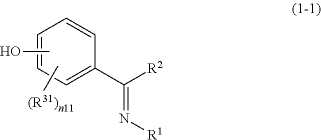
wherein R¹ represents a polymerizable group-containing hydrocarbon group optionally having a substituent; R² represents a hydrocarbon group optionally having a substituent; R³¹ represents a hydroxy group, a thiol group, a carboxy group, a sulfo group, a nitro group or a phosphate group; and n11 represents an integer of 0 to 4.

3. The filtering material according to claim 1, wherein the compound (1-0) is represented by general formula (1-2) shown below: 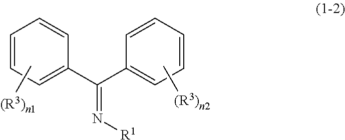
wherein R¹ represents a polymerizable group-containing hydrocarbon group optionally having a substituent; R³ represents a hydroxy group, a thiol group, a carboxy group, a sulfo group, a nitro group or a phosphate group; n1 represents an integer of 1 to 5, provided that the plurality of R³ may be the same or different from each other; and n2 represents an integer of 0 to 5.

4. The filtering material according to claim 1, wherein the compound (1-0) is represented by general formula (1-3) shown below: 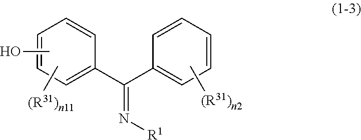
wherein R¹ represents a polymerizable group-containing hydrocarbon group optionally having a substituent; R³¹ represents a hydroxy group, a thiol group, a carboxy group, a sulfo group, a nitro group or a phosphate group, provided that the plurality of R³¹ may be the same or different from each other; n11 represents an integer of 0 to 4; and n2 represents an integer of 0 to 5.

5. A filter comprising the filtering material according to claim 1.

6. A method of filtration comprising:
a resist composition or a solvent through the filter according to claim 5; and
removing impurities contained in the resist composition or the solvent.

7. A method of producing a phenylimine compound, in which a compound (K) having an aromatic ring and a carbonyl group is reacted with a hydrocarbon compound (A) having at least one amino group, thereby obtaining the phenylimine compound represented by general formula (1-0) shown below, the aromatic ring having at least one group selected from the group consisting of a hydroxy group, a thiol group, a carboxy group, a sulfo group, a nitro group and a phosphate group: 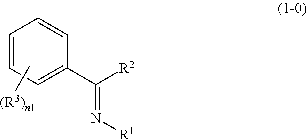
wherein R¹ represents a polymerizable group-containing hydrocarbon group optionally having a substituent; R² represents a hydrocarbon group optionally having a substituent; R³ represents a hydroxy group, a thiol group, a carboxy group, a sulfo group, a nitro group or a phosphate group; and n1 represents an integer of 1 to 5.

8. The method of producing a phenylimine compound according to claim 7, wherein the phenylimine compound is represented by general formula (1-1) shown below: 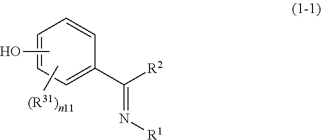
wherein R¹ represents a polymerizable group-containing hydrocarbon group optionally having a substituent; R² represents a hydrocarbon group optionally having a substituent; R³¹ represents a hydroxy group, a thiol group, a carboxy group, a sulfo group, a nitro group or a phosphate group; and n11 represents an integer of 0 to 4.

9. The method of producing a phenylimine compound according to claim 7, wherein the phenylimine compound is represented by general formula (1-3) shown below: 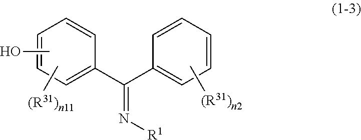
wherein R¹ represents a polymerizable group-containing hydrocarbon group optionally having a substituent; R³¹ represents a hydroxy group, a thiol group, a carboxy group, a sulfo group, a nitro group or a phosphate group, provided that the plurality of R³¹ may be the same or different from each other; n11 represents an integer of 0 to 4; and n2 represents an integer of 0 to 5.

10. The method of producing a phenylimine compound according to claim 8, wherein the compound (K) is represented by general formula (K1) shown below, and the hydrocarbon compound (A) is represented by general formula (A1) shown below: 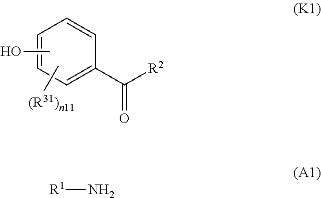
wherein R¹ represents a polymerizable group-containing hydrocarbon group optionally having a substituent; R² represents a hydrocarbon group optionally having a substituent; R³¹ represents a hydroxy group, a thiol group, a carboxy group, a sulfo group, a nitro group or a phosphate group; and n11 represents an integer of 0 to 4.

11. A method of producing an alkoxyphenylimine compound comprising reacting the phenylimine compound obtained by the method according to claim 7 with an alkylation agent.

12. The method of producing an alkoxyphenylimine compound according to claim 11, wherein the alkylation agent is one or more selected from the group consisting of dimethylsulfate, iodomethane, dimethylcarbonate and diethylsulfate.

13. A compound represented by general formula (1-1) shown below: 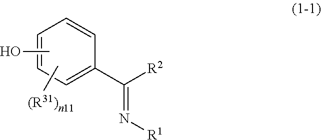
wherein R¹ represents a polymerizable group-containing hydrocarbon group optionally having a substituent; R² represents a hydrocarbon group optionally having a substituent; R³¹ represents a hydroxy group, a thiol group, a carboxy group, a sulfo group, a nitro group or a phosphate group; and n11 represents an integer of 0 to 4.

14. The compound according to claim 13, wherein the compound is represented by general formula (1-3) shown below: 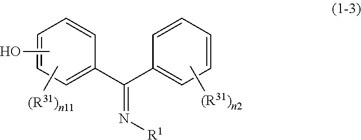
wherein R¹ represents a polymerizable group-containing hydrocarbon group optionally having a substituent; R³¹ represents a hydroxy group, a thiol group, a carboxy group, a sulfo group, a nitro group or a phosphate group, provided that the plurality of R³¹ may be the same or different from each other; n11 represents an integer of 0 to 4; and n2 represents an integer of 0 to 5.
